(19) United States
(12) Patent Application Publication
(10) Pub. No.: US 2025/0326985 A1
(43) Pub. Date: Oct. 23, 2025

(54) CYCLOHEXANE AND/OR CYCLOHEXENE ESTERS AS A FRAGRANCE

(71) Applicant: Symrise AG, Holzminden (DE)

(72) Inventors: Bernd HÖLSCHER, Halle (DE); Vijayanand CHANDRASEKARAN, Holzminden (DE)

(21) Appl. No.: 18/861,920

(22) PCT Filed: May 3, 2022
(86) PCT No.: PCT/EP2022/061831

§ 371 Date: Oct. 31, 2024

(51) Int. Cl. C11B 9/00; A61K 8/37; C07C 69/608

(52) U.S. Cl. CPC C11B 9/0034; A61K 8/37; C07C 69/608

(57) ABSTRACT

Suggested is the use of at least one compound of Formula I as a fragrance.

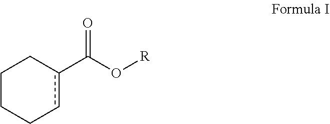

### FIELD OF THE INVENTION

The present invention relates to the use cyclohexane and/or cyclohexene esters as a fragrance. Furthermore, the present invention relates to fragrance compositions, their use and a method for imparting, modifying and/or enhancing certain fragrance notes.

### BACKGROUND OF THE INVENTION

On the part of the perfume industry, new fragrance creations are permanently needed. However, the creation of a new fragrance or fragrance compositions involves a number of challenges. For example, it is necessary to select a specific composition of a few fragrances from an almost unlimited number of possible structures known from the prior art, in order to satisfy a specific need of the market and provide a product that matches the specific profile required by the customer. Suitable fragrances show a very beneficial performance, but in combination with other fragrances may develop unpleasant and thus detrimental odor aspects, making it difficult or even impossible to use them for the specific purpose.

A second important problem concerns the need to provide fragrance compositions that are not only consistent with a particular odor profile, but also possess so-called secondary beneficial properties. Indeed, many fragrance compositions known from the market have significant drawbacks in use, such as poor solubility and stability to storage, but also failures in subjective issues such as richness, charisma, and the like. In addition, many known fragrance compositions require high dosages to achieve the desired odor result. Another requirement for fragrances today is high biodegradability as well as dermatological and toxicological safety. Consequently, there is a particularly high demand for providing fragrances that have a large effect on other fragrances even at small dosages and change rather unpleasant odor impressions into positive ones and/or enhance pleasant odor impressions.

Thus, the underlying problem of the present invention was to provide a new fragrance with the ability to enhance positive and beneficial fragrance aspects of other fragrances and/or (at the same time) to reduce, inhibit and/or mask undesirable unpleasant fragrance aspects. In particular, the new fragrance should also be characterized by improving the stability, solubility and overall performance of other fragrances, as well as reducing the required dosage. Finally, the fragrance compositions themselves should have excellent biodegradability and be harmless to humans and the environment.

### DESCRIPTION OF THE INVENTION

This is solved by the use of least one compound of Formula I

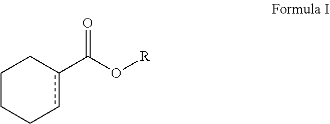

as a fragrance, wherein
R represents ethyl, propyl, butyl or cyclopentyl; and
the dotted line represents a carbon-carbon single bond or a carbon-carbon double bond.

The compounds according to Formula I to be used according to the invention may be present in any stereoisomeric form or may be present as any mixture of stereoisomers.

What has been said herein for compounds of Formula I, in particular the advantages described herein, also apply to a mixture of stereoisomers of compounds of Formula I to be used or to be employed according to the invention or to a mixture of different compounds of Formula I.

The compounds of Formula I possess olfactory properties which are quite unique and which clearly differ from and also surpass those of known odoriferous substances. The suitability of the compounds of Formula I as fragrances was previously unknown. It is therefore particularly surprising that in the already well-studied field a fragrance with valuable, interesting and complex olfactory properties could be found.

As mentioned above, the suitability as a fragrance for the compounds of Formula I is not known. Likewise, no precise odor description is available, however, according to the inventors, the compounds of Formula I smell in particular like cinnamon, marzipan, fruity, valerate, sweet, apple, pear, pineapple, strawberry, green and/or castoreum.

Consequently, the inventors have made the surprising discovery that the compounds of Formula I are suitable as a fragrance and, in small dosages, produce special effects in combination with other fragrances.

For the sake of clarity, by the expression “the dotted lines represent a carbon-carbon single bond or a carbon-carbon double bond”, or the similar, it is meant the normal meaning understood by a person skilled in the art, i.e. that the whole bonding (solid and dotted line) between the carbon atoms connected by said dotted line is a single or a double bond.

In a preferred embodiment according to the invention, R represents ethyl, butyl or cyclopentyl. In a further preferred embodiment according to the invention, R represents ethyl, butyl or cyclopentyl and the dotted line represents a carbon-carbon single bond. In a further preferred embodiment according to the invention, R represents ethyl, butyl or cyclopentyl and the dotted line represents carbon-carbon double bond.

Preferably, the compound of Formula I is ethyl cyclohexene-1-carboxylate which has a structure according to Formula II

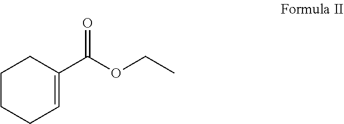

and/or a molecular weight of 154.1.

Preferably, ethyl cyclohexene-1-carboxylate has the following odor nores: cinnamon, marzipan, chlorine and/or benzophenone.

Further preferably, the compound of Formula I is butyl cyclohexenecarboxylate which has a structure according to Formula III

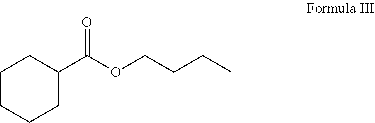

and/or a molecular weight of 184.14.

Preferably, butyl cyclohexenecarboxylate has the following odor notes: fruity, valerate, sweet, cinnamon, castoreum, apple, pear and/or pineapple.

Further preferably, the compound of Formula I is cyclopentyl cyclohexanecarboxylate which has a structure according to Formula IV

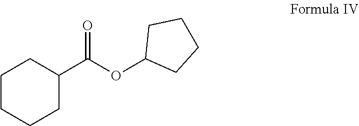 [SMILES: O=C(OC1CCCC1)C1CCCCC1]

and/or a molecular weight of 196.15

Preferably, cyclopentyl cyclohexanecarboxylate has the following odor notes: fruity, pineapple, green and/or strawberry.

Further preferably, the compound of Formula I is selected from the group consisting of ethyl cyclohexene-1-carboxylate, butyl cyclohexenecarboxylate, cyclopentyl cyclohexanecarboxylate or their mixtures.

In a preferred embodiment according to the invention, at least one compound of Formula I is used in a fragrance composition to obtain at least one of the following fragrance notes: cinnamon, marzipan, fruity, valerate, sweet, apple, pear, pineapple, strawberry, green and/or castoreum. Further preferred is the use of at least one compound of Formula I for imparting, modifying and/or enhancing a fragrance composition to make it more fruity, juicier, softer, clearer, fresher, sweeter and/or more floral. This is, however particularly surprising in light of the above odor description, since some compounds of Formula I do not have any fruity, juicy or floral note and/or even a less pleasant odor note, for example ethyl cyclohexene-1-carboxylate.

The fact that compounds of Formula I to be used according to the invention may impart a very complex and varied overall sensory impression, which can otherwise usually only be achieved by mixtures of several components (such as essential oils or spice mixtures), is particularly surprising.

Beyond the primary, namely olfactory, properties, compounds of Formula I additionally possess positive secondary properties, in particular a high substantivity compared to fragrances with similar olfactory properties, as well as a high stability in certain media and preparations, a high extensibility, and is also biodegradable.

In connection with the preferred use for imparting, modifying and/or enhancing a fragrance note, it is also recognized that compounds of Formula I can excellently function as a so-called booster (amplifier; enhancer).

Moreover, compounds of Formula I to be used according to the invention can enhance the intensity of a fragrance mixture (fragrance composition) and round off the overall odor of the mixture. The compounds described herein can therefore be used to impart more fullness, freshness, (radiance) power, radiance, roundness, harmony, naturalness and/or less greasiness to a fragrance composition, in particular the compound can be used to impart more softness, more clearness and/or more freshness to a fragrance composition.

Furthermore, the compounds of Formula I are suitable as an agent for increasing the substantivity and/or retention of a fragrance composition.

The compounds of Formula I can be used in a variety of products; they can be used as a single fragrance, but they can be combined particularly advantageously with other fragrances in different, different proportions to form fragrance mixtures, and novel and original perfume compositions can also be created. Accordingly, one aspect of the invention also relates to fragrance mixture and possibly further constituents (solvents or the like), which contains at least one compound of Formula I 4-cyclohexylbutan-2-one.

The fragrance composition according to the invention comprises at least one compound of Formula I

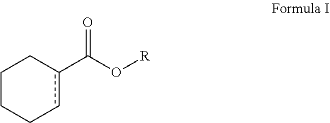

wherein
R represents ethyl, propyl, butyl or cyclopentyl; and
the dotted line represents a carbon-carbon single bond or a carbon-carbon double bond; and at least one further fragrance.

In a preferred embodiment, the compound of Formula I is selected from the group consisting of ethyl cyclohexene-1-carboxylate, butyl cyclohexenecarboxylate, cyclopentyl cyclohexanecarboxylate or their mixtures.

Preferably, the fragrance composition according to the invention comprises or consists of at least one compound of Formula I and two or more further fragrances. More preferably, the fragrance composition according to the invention comprises or consists of at least one compound of Formula I and three or more further fragrances. Most preferably, the fragrance composition according to the invention comprises or consists of at least one compound of Formula I and four or more further fragrances.

Preferably, the fragrance composition according to the invention comprises or consists of ethyl cyclohexene-1-carboxylate and two or more further fragrances. More preferably, the fragrance composition according to the invention comprises or consists of ethyl cyclohexene-1-carboxylate and three or more further fragrances. Most preferably, the fragrance composition according to the invention comprises or consists of ethyl cyclohexene-1-carboxylate and four or more further fragrances.

Preferably, the fragrance composition according to the invention comprises or consists of butyl cyclohexenecarboxylate and two or more further fragrances. More preferably, the fragrance composition according to the invention comprises or consists of butyl cyclohexenecarboxylate and three or more further fragrances. Most preferably, the fragrance composition according to the invention comprises or consists of butyl cyclohexenecarboxylate and four or more further fragrances.

Preferably, the fragrance composition according to the invention comprises or consists of cyclopentyl cyclohexanecarboxylate and two or more further fragrances. More preferably, the fragrance composition according to the invention comprises or consists of cyclopentyl cyclohexanecarboxylate and three or more further fragrances. Most preferably, the fragrance composition according to the invention comprises or consists of cyclopentyl cyclohexanecarboxylate and four or more further fragrances.

The compounds of Formula I according to the invention are usually used in sensory effective amounts, i.e. in a total amount in which they exert a sensory effect.

Preferably, the weight ratio of the at least one compound of Formula I to the total amount of other fragrances is in the range of 1:100 to 1:10. More preferably, the weight ratio of the at least one compound of Formula I to the total amount of other fragrances is in the range of 1:85 to 1:15.

In this context, it is preferred if the total amount of the at least one compound of Formula I is in the range from 0.0001 to 99.9% by weight, preferably from 0.001 to 99.5% by weight, particularly preferably from 0.01 to 99% by weight, from 0.01 to 90% by weight, from 0.01 to 50% by weight, from 0.01 to 20% by weight, from 0.01 to 10% by weight and particularly preferably from 0.01 to 5% by weight, in particular from 0.05 to 2.0% by weight, in each case based on the total weight of the composition

In a preferred embodiment according to the invention, the compounds of Formula I are contained in the fragrance composition in a sensory effective amount sufficient to modify an existing fragrance to make it more fruity, juicier, softer, clearer, fresher, sweeter and/or more floral.

Examples of fragrances which may advantageously be combined with the compounds of Formula I in the context of the present invention can be found, for example, in S. Arctander, Perfume and Flavor Materials, Vol. I and II, Montclair, N. J. 1969, self-published, or K. Bauer ei a/., Common Fragrance and Flavor Materials, 4th Edition, Wiley-VCH, Weinheim 2001.

Specifically mentioned are: Extracts from natural raw materials such as essential oils, concretes, absolutes, resins, resinoids, balsams, tinctures such as.
Ambergris tincture; Amyris oil; Angelica seed oil; Angelica root oil; Anise oil; Valerian oil; Basil oil; tree moss absolute; bay oil; mugwort oil; benzoeresin; bergamot oil; Beeswax absolue; Birch tar oil; Bitter almond oil; Savory oil; Bucco leaf oil; Cabreuva oil; Cade oil; Calmus oil; Camphor oil; Cananga oil; Cardamom oil; Cascarilla oil; Cassia oil; Cassie-absolue; Castoreum-absolue; Cedar leaf oil; Cedarwood oil; Cistus oil; Citronella oil; Citron oil; Copaiva balsam; Copaiva balsam oil; Coriander oil; Costus root oil; Cumin oil; Cypress oil; Davana oil; Dill herb oil; Dill seed oil; Eau de brouts-Absolue; Oak moss absolute; Elemi oil; Tarragon oil; Eucalyptus citriodora oil; Eucalyptus oil; Fennel oil; Spruce needle oil; Galbanum oil; Galbanum resin; Geranium oil; Grapefruit oil; Guaiac wood oil; Gurjun balsam; Gurjun balsam oil; Helichrysum absolute; Helichrysum oil; Ginger oil; Iris root absolute; Iris root oil; Jasmine absolute; Calamus oil; Chamomile oil blue; Chamomile oil roman; Carrot seed oil; Cascarilla oil; Pine needle oil; Spearmint oil; Caraway oil; labdanum oil; labdanum absolute; labdanum resin; lavandin absolute; Lavandin oil; Lavender absolute; Lavender oil; Lemongrass oil; Lovage oil; Lime oil distilled; Lime oil pressed; Linaloe oil; Litsea cubeba oil; Bay leaf oil; Mace oil; Marjoram oil; Mandarin oil; Massoir bark oil; Mimosa absolute; Musk grain oil; Musk tincture; Muscat sage oil; Nutmeg oil; Myrrh absolute; Myrrh oil; Myrtle oil; Clove leaf oil; Clove flower oil; Neroli oil; Olibanum absolute; Olibanum oil; Opopanax oil; orange blossom absolute; orange oil; origanum oil; palmarosa oil; patchouli oil; Perilla oil; Perubalsam oil; Parsley leaf oil; Parsley seed oil; Petitgrain oil; Peppermint oil; Pepper oil; Allspice oil; Pine oil; Poley oil; Rose absolute; Rosewood oil; Rose oil; Rosemary oil; Sage oil Dalmatian; Sage oil Spanish; Sandalwood oil; Celery seed oil; Spicy lavender oil; Star anise oil; Styrax oil; Tagetes oil; Fir needle oil; Tea tree oil; Turpentine oil; Thyme oil; Tolu balsam; Tonka absolue; tuberose-absolue; vanilla extract; violet leaf-absolue; verbena oil; vetiver oil; juniper berry oil; wine yeast oil; wormwood oil; wintergreen oil; ylang oil; hyssop oil; civet-absolue; cinnamon leaf oil; cinnamon bark oil and fractions thereof, or Ingredients isolated therefrom;

Individual odorants from the group of hydrocarbons, such as 3-carene; α-pinene; ß-pinene; a-terpinene; γ-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
of the aliphatic alcohols such as Hexanol; octanol; 3-octanol; 2,6-dimethylheptanol; 2-methyl-2-heptanol; 2-methyl-2-octanol; (E)-2-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
of aliphatic aldehydes and their acetals, such as Hexanal; heptanal; octanal; nonanal; decanal; undecanal; dodecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6, 10-trimethyl-9-undecenal; 2,6, 10-trimethyl-5,9-undecadienal; heptanaldiethyl acetal; 1, 1-dimethoxy-2,2,5-trimethyl-4-hexene; citronellyloxyacetaldehyde; 1-(1-methoxy-propoxy)-(E/Z)-3-hexene;
of aliphatic ketones and their oximes such as 2-heptanone; 2-octanone; 3-octanone; 2-nonanone; 5-methyl-3-heptanone; 5-methyl-3-heptanone oxime; 2,4,4,7-tetramethyl-6-octen-3-on; 6-methyl-5-hepten-2-one;
of aliphatic sulfur-containing compounds such as 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthen-8-thiol;
of aliphatic nitriles such as 2-nonenoic acid nitrile; 2-undecenoic acid nitrile; 2-tridecenoic acid nitrile; 3, 12-tridecadienoic acid nitrile; 3,7-dimethyl-2,6-octadienoic acid nitrile; 3,7-dimethyl-6-octenoic acid nitrile;
esters of aliphatic carboxylic acids, e.g. (E)- and (Z)-3-hexenyl formate; ethyl acetoacetate; isoamyl acetate; 3,5,5-trimethylhexyl acetate; 3-methyl-2-butenyl acetate; (E)-2-hexenyl acetate; (E)- and (Z)-3-hexenyl acetate; Octyl acetate; 3-octyl acetate; 1-octene-3-yl acetate; ethyl butyrate; butyl butyrate; isoamyl butyrate; hexyl butyrate; (E)- and (Z)-3-hexenyl isobutyrate; Hexyl crotonate; ethyl isovalerate; ethyl 2-methylpentanoate; ethyl hexanoate; allyl hexanoate; ethyl heptanoate; allyl heptanoate; ethyl octanoate; Ethyl (E,Z)-2,4-decadienoate; methyl 2-octinate; methyl 2-noninate; allyl 2-isoamyloxyacetate; Methy 1-3,7-dimethyl-2,6-octadiene-oate; 4-methyl-2-pentyl crotonate;
of acyclic terpene alcohols such as. Geraniol; nerol; lavadulol; nerolidol; farnesol; tetrahydrolinalool; tetrahydrogeraniol; 2,2-Dimethyl-3-(3-methylphenyl) propan-1-ol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-Dimethyloct-6-en-1-ol; (2E)-3,7-Dimethylocta-2,6-dien-1-ol; 3,7-dimethyl-1,5,7-octatrien-3-ol 2,6-dimethyl-2,5J-octatrien-1-ol; and their formates, acetates, propionates, isobutyrates, butyrates, isovalerianates, pentanoates, hexanoates, crotonates, tiglinates and 3-methyl-2-butenoates;
of the acyclic terpene aldehydes and ketones such as citronellal; 7-methoxy-3,7-dimethyloctanal; 2,6,10-trimethyl-9-undecenal; geranylacetone; and the dimethyl and diethylacetals of geranial, neral,
of cyclic terpene alcohols such as. menthol; isopulegol; alpha-terpineol; terpinenol-4; menthan-8-ol; menthan-1-ol; menthan-7-ol; borneol; isoborneol; linalool oxide; nopol; cedrol; ambrinol; vetiverol; guaiaol; and their formates, acetates, propionates, isobutyrates, butyrates, isovalerianates, pentanoates, hexanoates, crotonates, tiglinates and 3-methyl-2-butenoates;
of cyclic terpene aldehydes and ketones such as Menthone; isomenthone; 8-mercaptomenthan-3-on; carvone; camphor; fenchone; alpha-Ionone; beta-Ionone; alpha-n-methylionone; beta-n-methylionone; alpha-Isomethylionone; beta-Isomethyl-ionone; alpha-irone; beta-damascenone; 1-(2,4,4-trimethyl-2-cyclohexen-1-yl)-2-buten-1-one; 1,3,4,6,7,8a-Hexahydro-1, 1,5,5-tetramethyl-2H-2,4a-methano-naphthalen-8 (5H)-on; 2-Methyl-4-(2,6,6-trimethyl-1-cyclohexen-1-yl)-2-butenal; Nootkatone; dihydronootkatone; 4,6,8-megastigmatriene-3-on; alpha-sinensal; beta-sinensal; acetylated cedarwood oil (methylcedrylketone);
of cyclic alcohols such as 4-tert-butylcyclohexanol; 3,3,5-trimethylcyclohexanol; 3-Isocamphylcyclohexanol; 2,6,9-trimethyl-Z2,Z5,E9-cyclododecatrien-1-ol; 2-Isobutyl-4-m ethyltetrahydro-2H-pyran-4-ol; 4-cyclohexylbutan-2-ol;
of cycloaliphatic alcohols such as. alpha,3,3-trimethylcyclohexylmethanol; 1-(4-Isopropylcyclohexyl) ethanol; 2-methyl-4-(2,2,3-trimethyl-3-cyclopent-1-yl) butanol; 2-methyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-Methyl-5-(2,2,3-trimethyl-3-cyclopent-1-yl)-4-penten-2-ol; 3,3-Dimethyl-5-(2,2,3-trimethyl-3-cyclopent-1-yl)-4-penten-2-ol; 1-(2,2,6-Trimethylcyclohexyl) pentan-3-ol; 1-(2,2,6-Trimethylcyclohexyl) hexan-3-ol;
cyclic and cycloaliphatic ethers, e.g. Cineol; cedryl methyl ether; cyclododecyl methyl ether; 1, 1-dimethoxycyclododecane; (ethoxymethoxy)cyclo-dodecane; alpha-cedrene epoxide; 3a,6,6,9a-tetramethyldodecahydro-naphtho[2,1-b]furan; 3a-ethyl-6,6,9a-trimethyldodecahydronaphtho[2, 1 b]furan; 1,5,9-tri-methyl-13-oxabicyclo[10.1.0]trideca-4,8-diene; rose oxide; 2-(2,4-dimethyl-3-cyclohexen-1-yl)-5-methyl-5-(1-methyl propyl)-1, 3-dioxane;
of cyclic and macrocyclic ketones such as 4-tert.-butylcyclohexanone; 2,2,5-trimethyl-5-pentylcyclopentanone; 2-heptylcyclopentanone; 2-pentyl-cyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopenta-decanone; 4-(1-ethoxyvinyl)-3, 3,5,5-tetramethylcyclohexanone; 4-tert.-pentylcyclo-hexanone; 5-cyclohexadecen-1-one; 6,7-dihydro-1, 1,2,3,3-pentamethyl-4 (5H)-indanone; 8-cyclohexadecen-1-one; 9-cycloheptadecen-1-on; cyclopentadecanone; cyclohexadecanone;
of cycloaliphatic aldehydes such as 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
of cycloaliphatic ketones, e.g. 1-(3,3-Dimethylcyclohexyl)-4-penten-1-on; 2,2-Dimethyl-1-(2,4-dimethyl-3-cyclohexen-1-yl)-1-propanon; 1-(5,5-Dimethyl-1-cyclohexen-1-yl)-4-penten-1-on; 2, 3,8,8-tetramethyl-1, 2,3,4,5,6,7, 8-octahydro-2-naphthalenyl methyl ketone; methyl 2,6, 10-trimethyl-2,5,9-cyclododecatrienyl ketone; tert.-butyl-(2,4-dimethyl-3-cyclohexen-1-yl) ketone;
of ester of cyclic alcohols such as 2-tert-butylcyclohexyl acetate; 4-tert-butylcyclohexyl acetate; 2-tert-pentylcyclohexyl acetate; 4-tert-pentylcyclohexyl acetate; 3,3, 5-tri methylcyclohexyl acetate; decahydro-2-naphthyl acetate; 2-cyclo-pentylcyclopentyl crotonate; 3-pentyl tetrahydro-2H-pyran-4-yl acetate; decahydro-2,5,5,8a-tetramethyl-2-naphthyl acetate; 4,7-methano-3a,4,5,6,7,7a-hexa-hydro-5, resp. 6-indenyl acetate; 4,7-methano-3a,4,5,6,7,7a-hexahydro-5, or 6-indenyl propionate; 4,7-methano-3a,4,5,6,7,7a-hexahydro-5, or 6-indenyl isobutyrate; 4,7-methanooctahydro-5, or 6-indenyl acetate;
of esters of cycloaliphatic alcohols such as 1-cyclohexylethyl crotonate;
of esters of cycloaliphatic carboxylic acids, e.g. Allyl 3-cyclohexyl propionate; allyl cyclohexyloxy acetate; ice and trans-methyl dihydrojasmonate; ice and trans-methyl jasmonate; methyl 2-hexyl-3-oxocyclopentane carboxylate; ethyl 2-ethyl-6,6-dimethyl-2-cyclohexene carboxylate; ethyl 2,3,6,6-tetramethyl-2-cyclohexene carboxylate; ethyl 2-methyl-1,3-dioxolane-2-acetate;
of araliphatic alcohols such as Benzyl alcohol; 2-phenylethanol; 1-phenylethyl alcohol; 3-phenylpropanol; 2-phenylpropanol; 2-phenoxyethanol; 2,2-dimethyl-3-phenylpropanol; 2,2-dimethyl-3-(3-methylphenyl) propanol; 1-1-dimethyl-2-phenylethyl alcohol; 1, 1-dimethyl-3-phenylpropanol; 1-ethyl-1-methyl-3-phenyl-propanol; 2-methyl-5-phenylpentanol; 3-methyl-5-phenylpentanol; 3-phenyl-2-propen-1-ol; 4-methoxybenzyl alcohol; 1-(4-Isopropylphenyl) ethanol;
of esters of araliphatic alcohols and aliphatic carboxylic acids, e.g. Benzyl acetate; benzyl propionate; benzyl isobutyrate; benzyl isovalerate; 2-phenylethyl acetate; 2-phenylethyl propionate; 2-phenylethyl isobutyrate; 2-phenyl ethyl isovalerate; 1-phenylethyl acetate; alpha-trichloromethyl benzyl acetate; alpha, alpha-dimethylphenyl ethyl acetate; alpha, alpha-dimethylphenyl ethyl butyrate; cinnamyl acetate; 2-phenoxyethyl isobutyrate; 4-methoxybenzyl acetate;
of araliphatic ethers such as. 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetra-hydroindeno[1,2-d]-m-dioxin; 4,4a,5,9b-tetrahydro-2,4-dimethylindeno[1,2-d]-m-dioxin; of aromatic and araliphatic aldehydes such as. e.g., benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl) propanal; 2-methyl-3-(4-isobutylphenyl) propanal; 3-(4-tert. butyl-phenyl) propanal; cinnamaldehyde; alpha-butylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl) propanal; 2-methyl-3-(4-methylenedioxyphenyl) propanal;
of aromatic and araliphatic ketones such as acetophenone; 4-methylacetophenone; 4-methoxyacetophenone; 4-tert.-butyl-2,6-dimethylaceto-phenone; 4-phenyl-2-butanone; 4-(4-hydroxyphenyl)-2-butanone; 1-(2-naphtha-lenyl) ethanone; 2-benzofuranylethanone; (3-methyl-2-benzofuranyl) ethanone; benzophenone; 1, 1, 2,3,6-hexamethyl-5-indanyl methyl ketone; 6-tert.butyl-1,1-di-methyl-4-indanyl methyl ketone; 1-[2,3-dihydro-1, 1,2,6-tetramethyl-3-(1-methylethyl)-1H-5-indenyl]ethanone; 5′,6′,7′,8′-tetrahydro-3′,5′,5′,6′,8′,8′-hexamethyl-2-aceto-naphthone;
of aromatic and araliphatic carboxylic acids and their esters, e.g. Benzoic acid; phenylacetic acid; methyl benzoate; ethyl benzoate; hexyl benzoate; benzyl benzoate; methyl phenyl acetate; ethyl phenyl acetate; geranyl phenyl acetate; phenyl ethyl phenyl acetate; methyl cinnamate; ethyl cinnamate; benzyl cinnamate; phenyl ethyl cinnamate; cinnamyl cinnamate; allyl phenoxy acetate; methyl salicylate; hexyl salicylate; cyclohexyl salicylate; cis-3-hexenyl salicylate; benzyl salicylate; phenyl ethyl salicylate; methyl 2,4-dihydroxy-3,6-dimethylbenzoate; ethyl 3-phenyl glycidate; ethyl 3-methyl-3-phenyl glycidate;
of heterocyclic compounds such as 2,5-dimethyl-4-hydroxy-2H-furan-3-one; 2-ethyl-4-hydroxy-5-methyl-2H-fu ran-3-on; 3-hydroxy-2-methyl-4H-pyran-4-one; 2-ethyl-3-hydroxy-4H-pyran-4-one;
of lactones such as. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1, 15-pentadecanolide; 1, 16-hexadecanolide; 9-hexadecene-1,16-olide; 10-oxa-1,16-hexadecanolide; 1 1-oxa-1,16-hexa-decanolide; 12-oxa-1,16-hexadecanolide; ethylene-1,12-dodecanedioate; ethylene-1, 13-tridecanedioate; 2,3-dihydrocoumarin; octahydrocoumarin.

In a preferred embodiment according to the invention, the at least one further fragrance is selected from the group consisting of AGRUMEX LC, ALDEHYD C10, ALDEHYD C11 UNDECANAL, ALDEHYD C12 LAURIN, ALDEHYD C12 MNA, ALDEHYDE C11 MOA PURE, ALLYLCYCLOHEXYLPROPIONAT, ALLYLHEPTYLAT, AMBROXIDE 10% IPM, ANISALDEHYD REIN, CALONE 10%, CARVON L, CUMARIN, CYCLODUMOL ACETATE, 2,6-Dimethyl-7-octen-2-ol (DIHYDROMYRCENOL), DYNASCONE 1% DPG, FARENAL® 10% DPG, FLORAZON, FLORHYDRAL, FLOROSA BM/PYRANOL, FRUCTATE®, FURAMINTON/TONKALACTONE™ 1% DPG, GLOBALIDE®, Methyl-3-oxo-2-pentyl-1-cyclopentylacetat (HEDION), HELIOTROPIN/PIPERONAL, HEXYLACETAT, HEXYLSALICYLAT, ISOAMYLACETAT, KRAUSEMINZOEL 65% AMERIK., LEAFOVERT®, LINOLAL, MAGNOLAN, MELONAL, METHYLNAPHTHYLKETON BETA KRIST., NEROLIONE 0.1% DPG, 2-phenylethanol (PHENYLETHYLALKOHOL), TIMBERSILK, VERTOMUGAL, ACETESSIGSAEUREETHYLESTER, ALDEHYD C14 SOG, ALDEHYD C18 SOG., ALLYLCAPRONAT, BENZALDEHYD DD, CITRAL FF, CITRONELLOL 950, CITRONELLYLACETAT EXTRA, CITRONENOEL ITAL., CITRONENOEL TERPENE, CYCLAMENALDEHYD, DAMASCENON, DAMASCONE DELTA, DECALACTON GAMMA, _ETHYLBUTYRAT, ETHYLMALTOL, ETHYLMETHYLBUTYRAT-2, GERANYLACETAT PUR, HELIOTROPIN/PIPERONAL, HERBAFLORAT, HEXENOL CIS-3, HEXENYLACETAT CIS-3, IONON BETA, ISO E SUPER, ISOBORNEOL, ISOPROPYLMETHYLTHIAZOL-2,4 10% DPG, JASMAPRUNAT, LINALOOL, MANZANATE, OCTALACTON GAMMA, STYROLYLACETAT, SYMFRESH® NX, THIOMENTHANON-8,3 10% DPG, UNDECAVERTOL, VANILLIN, VERTOCITRAL, _ALLYLAMYLGLYCOLAT, AMYLZIMTALDEHYD ALPHA, BENZYLACETAT, DIMETHYLBENZYLCARBINYLBUTYRAT, DODECALACTON GAMMA, ETHYLENBRASSYLAT, ETHYLLINALOOL, ETHYLVANILLIN, FLOROPAL, FRAMBINON®, HERBYLPROPIONAT, HEXYLZIMTALDEHYD ALPHA, ISOAMYLACETAT, ISOAMYLBUTYRAT, JASMAPRUNAT, MACROLIDE® SUPRA, METHYLANTHRANILAT, METHYLCINNAMAT, MUGETANOL, PHENIRAT®, PRENYLACETAT, ROMANDOLIDE.

Preferably, at least one compound of Formula I is combined with one or more, particularly preferably with two, three, four, five or more of those preferred other fragrances.

Perfume oil compositions (=fragrance mixtures) containing at least one compound of Formula I are advantageously used for perfuming in liquid form, undiluted or diluted with a solvent. Suitable solvents for this are e.g. Ethanol, isopropanol, diethylene glycol monoethyl ether, glycerin, propylene glycol, 1, 2-butylene glycol, dipropylene glycol, diethyl phthalate, triethyl citrate, isopropyimyristate etc.

Furthermore, fragrance compositions according to the invention can be adsorbed on a carrier, which ensures both a fine distribution of the fragrances in the product and a controlled release during use. Such carriers can be porous inorganic materials such as light sulfate, silica gels, zeolites, plasters, clays, clay granules, aerated concrete, etc. or organic materials such as wood, cellulose-based substances, sugar, dextrins (e.g. maltodextrin) or plastics such as PVC, polyvinyl acetates or polyurethanes. The resulting combination of compositions according to the invention and carrier substance is also to be understood as a fragrance composition according to the invention.

Fragrance compositions according to the invention can also be microencapsulated, spray-dried, as inclusion complexes or as extrusion products and added in this form to a product to be perfumed, for example.

If necessary, the properties of the compositions modified in this way can be further optimized by so-called “coating” with suitable materials with a view to a more targeted release of fragrances, for which purpose waxy plastics such as polyvinyl alcohol are preferably used. The resulting products in turn represent articles according to the invention.

A further aspect of the present invention relates to the use of the fragrance composition to modify an existing fragrance to make it modify an existing fragrance to make it more fruity, juicier, softer, clearer, fresher, sweeter and/or more floral. Preferred embodiments listed above also apply to the use of the fragrance composition.

Fragrance compositions according to the invention can advantageously be used in concentrated form, in solutions or in the modified form described above for the production of perfumed articles according to the invention, such as. B. perfume extracts, eau de perfumes, eau de toilettes, aftershave, eau de colognes, pre-shave products, splash colognes and perfumed refreshing towels and the perfuming of acidic, alkaline and neutral cleaning agents such as floor cleaners, window glass cleaners, dishwashing detergents, Bathroom and sanitary cleaners, scouring milk, solid and liquid toilet cleaners, powder and foam carpet cleaners, textile fresheners, ironing aids, liquid detergents, powder detergents, laundry pretreatment agents such as bleaches, soaking agents and stain removers, fabric softeners, laundry soaps, washing tablets, and disinfectants Air fresheners in liquid, gel-like or applied form on a solid carrier, aerosol sprays, waxes and polishes such as furniture polishes, floor waxes, shoe creams and personal care products such as solid and liquid soaps, shower gels, shampoos, shaving soaps, shaving foams s, bath oils, cosmetic emulsions of the oil-in-water, of the water-in-oil and of the water-in-oil-in-water type such as, for example, skin creams and lotions, face creams and lotions, sun protection creams and lotions, after-sun creams and lotions, hand creams and lotions, foot creams and lotions, depilatory creams and lotions, after-shave creams and lotions, tanning creams and lotions, hair care products such as hair sprays, hair gels, setting hair lotions, Hair conditioners, permanent and semi-permanent hair dyes, hair shaping agents such as cold waves and hair straighteners, hair lotions, hair creams and lotions, deodorants and antiperspirants such as underarm sprays, roll-ons, deodorant sticks, deodorant creams, products in decorative cosmetics such as eye shadow, nail varnish, Lipsticks, mascara and candles, lamp oils, incense sticks, insecticides, repellants and fuels.

Of course, fragrance compositions according to the invention can also be included in cosmetic compositions or household compositions.

Another aspect of the invention relates to a method for modifying an existing fragrance to make it more fruity, juicier, softer, clearer, fresher, sweeter and/or more floral,
comprising or consisting of the following steps:
(a) providing at least one compound of Formula I

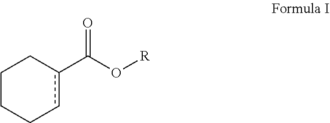

wherein
R represents ethyl, propyl, butyl or cyclopentyl; and
the dotted line represents a carbon-carbon single bond or a carbon-carbon double bond; and
(b) providing at least one further fragrance,
(c) adding the at least one compound of Formula (I) to the at least one further fragrance in a sensory effective amount sufficient to modify an existing fragrance to make it more more fruity, juicier, softer, clearer, fresher, sweeter and/or more floral.

Preferably, the weight ratio of the at least one compound of Formula I to the total amount of other fragrances is in the range of 1:100 to 1:10. More preferably, the weight ratio of the at least one compound of Formula I to the total amount of other fragrances is in the range of 1:85 to 1:15.

In this context, it is preferred if the total amount of the at least one compound of Formula I is in the range from 0.0001 to 99.9% by weight, preferably from 0.001 to 99.5% by weight, particularly preferably from 0.01 to 99% by weight, from 0.01 to 90% by weight, from 0.01 to 50% by weight, from 0.01 to 20% by weight, from 0.01 to 10% by weight and particularly preferably from 0.01 to 5% by weight, in particular from 0.05 to 2.0% by weight, in each case based on the total weight of the composition

In a preferred embodiment according to the invention, the at least one further fragrance is selected from the group consisting of AGRUMEX LC, ALDEHYD C10, ALDEHYD C11 UNDECANAL, ALDEHYD C12 LAURIN, ALDEHYD C12 MNA, ALDEHYDE C11 MOA PURE, ALLYLCYCLOHEXYLPROPIONAT, ALLYLHEPTYLAT, AMBROXIDE 10% IPM, ANISALDEHYD REIN, CALONE 10%, CARVON L, CUMARIN, CYCLODUMOL ACETATE, 2,6-Dimethyl-7-octen-2-ol (DIHYDROMYRCENOL), DYNASCONE 1% DPG, FARENAL® 10% DPG, FLORAZON, FLORHYDRAL, FLOROSA BM/PYRANOL, FRUCTATE®, FURAMINTON/TONKALACTONE™ 1% DPG, GLOBALIDE®, Methyl-3-oxo-2-pentyl-1-cyclopentylacetat (HEDION), HELIOTROPIN/PIPERONAL, HEXYLACETAT, HEXYLSALICYLAT, ISOAMYLACETAT, KRAUSEMINZOEL 65% AMERIK., LEAFOVERT®, LINOLAL, MAGNOLAN, MELONAL, METHYLNAPHTHYLKETON BETA KRIST., NEROLIONE 0.1% DPG, 2-phenylethanol (PHENYLETHYLALKOHOL), TIMBERSILK, VERTOMUGAL, ACETESSIGSAEUREETHYLESTER, ALDEHYD C14 SOG, ALDEHYD C18 SOG., ALLYLCAPRONAT, BENZALDEHYD DD, CITRAL FF, CITRONELLOL 950, CITRONELLYLACETAT EXTRA, CITRONENOEL ITAL., CITRONENOEL TERPENE, CYCLAMENALDEHYD, DAMASCENON, DAMASCONE DELTA, DECALACTON GAMMA, _ETHYLBUTYRAT, ETHYLMALTOL, ETHYLMETHYLBUTYRAT-2, GERANYLACETAT PUR, HELIOTROPIN/PIPERONAL, HERBAFLORAT, HEXENOL CIS-3, HEXENYLACETAT CIS-3, IONON BETA, ISO E SUPER, ISOBORNEOL, ISOPROPYLMETHYLTHIAZOL-2,4 10% DPG, JASMAPRUNAT, LINALOOL, MANZANATE, OCTALACTON GAMMA, STYROLYLACETAT, SYMFRESH® NX, THIOMENTHANON-8,3 10% DPG, UNDECAVERTOL, VANILLIN, VERTOCITRAL, _ALLYLAMYLGLYCOLAT, AMYLZIMTALDEHYD ALPHA, BENZYLACETAT, DIMETHYLBENZYLCARBINYLBUTYRAT, DODECALACTON GAMMA, ETHYLENBRASSYLAT, ETHYLLINALOOL, ETHYLVANILLIN, FLOROPAL, FRAMBINON®, HERBYLPROPIONAT, HEXYLZIMTALDEHYD ALPHA, ISOAMYLACETAT, ISOAMYLBUTYRAT, JASMAPRUNAT, MACROLIDE® SUPRA, METHYLANTHRANILAT, METHYLCINNAMAT, MUGETANOL, PHENIRAT®, PRENYLACETAT, ROMANDOLIDE.

Preferably, the at least one compound of Formula I is combined with one or more, particularly preferably with two, three, four, five or more of those preferred other fragrances.

Further preferably, the compound of Formula I is selected from the group consisting of ethyl cyclohexene-1-carboxylate, butyl cyclohexenecarboxylate, cyclopentyl cyclohexanecarboxylate or their mixtures.

Further preferred embodiments listed above also apply to method of the present invention.

A further aspect of the present invention relates to a perfumed product comprising at least one compound of Formula I

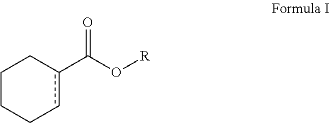

wherein
R represents ethyl, propyl, butyl or cyclopentyl; and
the dotted line represents a carbon-carbon single bond or a carbon-carbon double bond; and
two or more other fragrances and optionally other ingredients, characterized in that the product comprises the at least one compound of Formula (I) in a quantity which modifies the olfactory properties of the two or more other fragrances to make it more fruity, juicier, softer, clearer, fresher, sweeter and/or more floral.

In a preferred embodiment the perfumed product is selected from cosmetic, hygiene and/or household articles. Further preferred embodiments listed above also apply to the perfumed product of the present invention.

Preferably, the perfumed household article is selected from the group consisting of detergents and cleaning agents, hygiene or care products, preferably in the field of body and hair care, cosmetics and household, preferably from the group consisting of perfume extracts, Eau de perfumes, eau de toilets, aftershave lotions, eau de colognes, pre-shave products, splash colognes, perfumed refreshing tissues, acidic, alkaline or neutral cleaning agents, textile fresheners, ironing aids, liquid detergents, powder detergents, laundry pretreatment agents, fabric softeners, Wash tablets, disinfectants, surface disinfectants, air fresheners, aerosol sprays, waxes and polishes, personal care products, hand creams and lotions, foot creams and lotions, depilatory creams and lotions, after shave creams and lotions, tanning creams and lotions, hair care products, hair care products, Products of the decorative cosmetics, candles, lamp oils, incense sticks, insecticides, repellants and fuels. Most preferably, the perfumed product is selected from alcoholic perfume, a personal hygiene product or a cleaning or care product for use in the home.

In addition, it is preferred for the perfumed product according to the invention that at least one compound of Formula II is contained in a sensory effective amount which is sufficient for a consumer to have one or more olfactory properties selected from the group consisting of more fruity, juicier, softer, clearer, fresher, sweeter and/or more floral.

It is also preferred that the total amount the compounds of Formula I, based on the total weight of the perfumed product, is in the range from 0.00001 to 10% by weight, preferably from 0.0001 to 5% by weight %, particularly preferably 0.001 to 2% by weight, more preferably 0.5 to 2% by weight.

The additives, auxiliaries and/or active substances described above are preferably not odoriferous substances. These can be, for example, preservatives, antibacterial agents, chelating agents, cleaning agents, emulsifiers, fats, etc. and in principle all substances that are used as additives, auxiliaries and/or active ingredients in cosmetics, especially in fragrance compositions, as well as in household compositions.

Further preferred embodiments listed above also apply to the perfumed product of the present invention.

In the following, the invention is further characterized on the basis of the examples.

### EXAMPLES

### Example 1: Synthesis of ethyl cyclohexene-1-carboxylate

A mixture of cyclohexene-1-carboxylic acid (2.5 g, 19.2 mmol), ethanol (5.6 mL) and p-toluene sulfonic acid (0.4 g) in toluene (10 mL) was refluxed in Dean-Stark apparatus until no more water is distilled. After completion of the reaction, organic layer washed to neutral, evaporated in rotatory evaporator to get crude, followed by bulb-to-bulb distillation gave Wt=2.5 g, Y=66.6% of title compound.

2.5 g of cyclohexane-1-carboxylic acid gave 1.1 g, y=98% of

¹H NMR (400 MHZ, CDCl₃) δ 6.98 (tt, J=3.8, 1.8 Hz, 1H), 4.18 (q, J=7.1 Hz, 2H), 2.29-2.22 (m, 2H), 2.22-2.14 (m, 2H), 1.70-1.60 (m, 2H), 1.65-1.56 (m, 2H), 1.29 (t, J=7.1 Hz, 3H).

¹³C NMR (101 MHZ, CDCl₃) δ 167.71, 139.44, 130.50, 60.17, 25.76, 24.14, 22.11, 21.50, 14.31.

Odor: cinnamon, marzipan, chlorine, benzophenone

### Example 2: Synthesis of Butyl Cyclohexenecarboxylate

A mixture of cyclohexane carboxylic acid (400.0 g, 3.1 mol), butan-1-ol (418.4 g, 5.6 mol) and p-toluene sulfonic acid (2.5 g) in toluene (600 mL) was refluxed in Dean-Stark apparatus until no more water is distilled. After completion of the reaction, organic layer washed to neutral, evaporated in rotatory evaporator to get crude (571.0 g), followed by bulb-to-bulb distillation gave wt=467.0 g, Y=98% of butyl cyclohexenecarboxylate.

1H NMR (400 MHZ, CDCl₃) δ 4.06 (t, J=6.6 Hz, 2H), 2.28 (tt, J=11.3, 3.6 Hz, 1H), 1.95-1.84 (m, 2H), 1.80-1.69 (m, 2H), 1.69-1.62 (m, 1H), 1.60 (p, J=7.5, 6.6 Hz, 2H), 1.51-1.35 (m, 2H), 1.38 (h, J=7.5 Hz, 2H), 1.34-1.17 (m, 3H), 0.93 (t, J=7.4 Hz, 3H).

13C NMR (101 MHZ, CDCl₃) δ 176.26, 63.97, 43.32, 30.76, 29.08, 29.08, 25.82, 25.50, 25.50, 19.18, 13.74.

MS: 184, 168, 129, 111, 83, 55, 41, 29.

Odor: fruity, valerate, sweet, cinnamon, castoreum, apple, pear, pineapple

### Example 3: Synthesis of Cyclopentyl Cyclohexanecarboxylate

A mixture of cyclohexane carboxylic acid (400.0 g, 3.1 mol), cyclopentanol (304.0 g, 3.5 mol) and p-toluene sulfonic acid (4.0 g) in toluene (800 mL) was refluxed in Dean-Stark apparatus until no more water is distilled. After completion of the reaction, organic layer washed to neutral, evaporated in rotatory evaporator to get crude (629.3 g), followed by bulb-to-bulb distillation gave wt=551.6 g, Y=97% of cyclopentyl cyclohexanecarboxylate.

1H NMR (400 MHZ, CDCl₃) δ 5.15 (tt, J=5.7, 2.7 Hz, 1H), 2.23 (tt, J=11.2, 3.6 Hz, 1H), 1.92-1.77 (m, 4H), 1.77-1.72 (m, 2H), 1.72-1.65 (m, 2H), 1.65-1.50 (m, 5H), 1.49-1.35 (m, 2H), 1.33-1.22 (m, 2H), 1.27-1.14 (m, 1H).

13C NMR (101 MHZ, CDCl₃) δ 175.98, 76.51, 43.35, 32.71, 32.71, 29.01, 29.01, 25.83, 25.47, 25.47, 23.77, 23.77.

MS: 196, 168, 152, 129, 111, 83, 69, 55, 41, 29

Odor: fruity, pineapple, green, strawberry

### Example 4: Fragrances Mixtures

TABLE 1
Fragrance mixture 1

| AQUA | Ad 1000 | Ad 1000 | Ad 1000 |
|---|---|---|---|
| _AGRUMEX LC | 50 | 50 | 50 |
| _ALDEHYD C10 | 5 | 5 | 5 |
| _ALDEHYD C11 UNDECANAL | 2 | 2 | 2 |
| _ALDEHYD C12 LAURIN | 20 | 20 | 20 |
| _ALDEHYD C12 MNA | 30 | 30 | 30 |
| _ALDEHYDE C11 MOA PURE | 5 | 5 | 5 |
| _ALLYLCYCLOHEXYLPROPIONAT | 3 | 3 | 3 |
| _ALLYLHEPTYLAT | 10 | 10 | 10 |
| _AMBROXIDE 10% IPM | 5 | 5 | 5 |
| _ANISALDEHYD REIN | 5 | 5 | 5 |
| _CALONE 10% DPG | 15 | 15 | 15 |
| _CARVON L | 15 | 15 | 15 |
| _CUMARIN | 5 | 5 | 5 |
| _CYCLODUMOL ACETATE | 100 | 100 | 100 |
| _DIHYDROMYRCENOL | 150 | 150 | 150 |
| _DYNASCONE 1% DPG | 5 | 5 | 5 |
| _FARENAL ® 10% DPG | 15 | 15 | 15 |
| _FLORAZON | 15 | 15 | 15 |
| _FLORHYDRAL | 10 | 10 | 10 |
| _FLOROSA BM/PYRANOL | 150 | 150 | 150 |
| _FRUCTATE ® | 5 | 5 | 5 |
| _FURAMINTON/TONKALACTONE ™ 1% DPG | 5 | 5 | 5 |
| _GLOBALIDE ® | 15 | 15 | 15 |
| _HEDION | 20 | 20 | 20 |
| _HELIOTROPIN/PIPERONAL | 5 | 5 | 5 |
| _HEXYLACETAT | 20 | 20 | 20 |
| _HEXYLSALICYLAT | 50 | 50 | 50 |
| _ISOAMYLACETAT | 5 | 5 | 5 |
| _KRAUSEMINZOEL 65% AMERIK. | 5 | 5 | 5 |
| _LEAFOVERT ® | 15 | 15 | 15 |
| _LINOLAL | 10 | 10 | 10 |
| _MAGNOLAN | 20 | 20 | 20 |
| _MELONAL | 5 | 5 | 5 |
| _METHYLNAPHTHYLKETON BETA KRIST. | 10 | 10 | 10 |
| _NEROLIONE 0.1% DPG | 5 | 5 | 5 |
| _PHENYLETHYLALKOHOL | 30 | 30 | 30 |
| _TIMBERSILK | 30 | 30 | 30 |
| _VERTOMUGAL | 10 | 10 | 10 |
| DIPROPYLENGLYCOL | 20 | | |
| butyl cyclohexanecarboxylate | | 20 | |
| cyclopentyl cyclohexanecarboxylate | | | 20 |
| | citrus, | more fruity, | more soft |
| | watery | juicy and | and floral |
| | | pineapple | notes |

TABLE 2
Fragrance mixture 2

| | | |
|---|---|---|
| DIPROPYLENGLYCOL | 19 | 14 |
| _ACETESSIGSAEUREETHYLESTER | 3 | 3 |
| _AGRUMEX LC | 28 | 28 |
| _ALDEHYD C10 | 1 | 1 |
| _ALDEHYD C14 SOG | 28 | 28 |
| _ALDEHYD C18 SOG. | 1 | 1 |
| _ALLYLCAPRONAT | 7 | 7 |
| _ALLYLHEPTYLAT | 11 | 11 |
| _BENZALDEHYD DD | 0.5 | 0.5 |
| _CITRAL FF | 35 | 35 |
| _CITRONELLOL 950 | 1 | 1 |
| _CITRONELLYLACETAT EXTRA | 1 | 1 |
| _CITRONENOEL ITAL. | 15 | 15 |
| _CITRONENOEL TERPENE | 100 | 100 |
| _CYCLAMENALDEHYD | 1 | 1 |
| _DAMASCENON | 1 | 1 |
| _DAMASCONE DELTA | 3 | 3 |
| _DECALACTON GAMMA | 5 | 5 |
| _DIHYDROMYRCENOL | 14 | 14 |
| _ETHYLBUTYRAT | 1 | 1 |
| _ETHYLMALTOL | 0.5 | 0.5 |
| _ETHYLMETHYLBUTYRAT-2 | 7 | 7 |
| _GERANYLACETAT PUR | 0.5 | 0.5 |
| _HELIOTROPIN/PIPERONAL | 1 | 1 |
| _HERBAFLORAT | 10 | 10 |
| _HEXENOL CIS-3 | 3 | 3 |
| _HEXENYLACETAT CIS-3 | 5 | 5 |
| _HEXYLACETAT | 5 | 5 |
| _IONON BETA | 3 | 3 |
| _ISO E SUPER | 10 | 10 |
| _ISOAMYLACETAT | 0.5 | 0.5 |
| _ISOBORNEOL | 0.5 | 0.5 |
| _ISOPROPYLMETHYLTHIAZOL-2,4 10% DPG | 2 | 2 |
| _JASMAPRUNAT | 5 | 5 |
| _LINALOOL | 35 | 35 |
| _MANZANATE | 2 | 2 |
| _MELONAL | 1 | 1 |
| _OCTALACTON GAMMA | 0.5 | 0.5 |
| _PHENYLETHYLALKOHOL | 5 | 5 |
| _STYROLYLACETAT | 1 | 1 |
| _SYMFRESH ® NX | 20 | 20 |
| _THIOMENTHANON-8,3 10% DPG | 2 | 2 |
| _UNDECAVERTOL | 1 | 1 |
| _VANILLIN | 0.5 | 0.5 |
| _VERTOCITRAL | 3.5 | 3.5 |
| cyclopentyl cyclohexanecarboxylate | | 5 |

According to an expert panel, the fragrance mixture without cyclopentyl cyclohexanecarboxylate smells fruity with mango, peach, pineapple and citrus. The addition of cyclopentyl cyclohexanecarboxylate to the fragrances mixture makes the fragrance mixture clearer, fresher and juicier.

TABLE 3
Fragrance mixture 3

| | | | |
|---|---|---|---|
| DIPROPYLENGLYCOL | 80 | 60 | 40 |
| MATCHA GREEN | | | |
| _AGRUMEX LC | 150 | 150 | 150 |
| _ALDEHYD C14 SOG | 75 | 75 | 75 |
| _ALLYLAMYLGLYCOLAT | 1.5 | 1.5 | 1.5 |
| _ALLYLCAPRONAT | 1 | 1 | 1 |
| _ALLYLCYCLOHEXYLPROPIONAT | 20 | 20 | 20 |
| _ALLYLHEPTYLAT | 32 | 32 | 32 |
| _AMBROXIDE | 1 | 1 | 1 |
| _AMYLZIMTALDEHYD ALPHA | 15 | 15 | 15 |
| _BENZYLACETAT | 20 | 20 | 20 |
| _DAMASCONE DELTA | 2 | 2 | 2 |
| _DECALACTON GAMMA | 20 | 20 | 20 |
| _DIHYDROMYRCENOL | 50 | 50 | 50 |
| _DIMETHYLBENZYLCARBINYLBUTYRAT | 32 | 32 | 32 |
| _DIPROPYLENGLYCOL | 19.5 | 19.5 | 19.5 |
| _DODECALACTON GAMMA | 2.5 | 2.5 | 2.5 |
| _DYNASCONE | 0.5 | 0.5 | 0.5 |
| _ETHYLENBRASSYLAT | 90 | 90 | 90 |
| _ETHYLLINALOOL | 12 | 12 | 12 |
| _ETHYLMALTOL | 2 | 2 | 2 |
| _ETHYLMETHYLBUTYRAT-2 | 6 | 6 | 6 |
| _ETHYLVANILLIN | 5 | 5 | 5 |
| _FLOROPAL | 2 | 2 | 2 |
| _FLOROSA BM/PYRANOL | 22 | 22 | 22 |
| _FRAMBINON ® | 1.5 | 1.5 | 1.5 |
| _GLOBALIDE ® | 65 | 65 | 65 |
| _HERBYLPROPIONAT | 15 | 15 | 15 |
| _HEXENOL CIS-3 | 5 | 5 | 5 |
| _HEXENYLACETAT CIS-3 | 6 | 6 | 6 |
| _HEXYLACETAT | 20 | 20 | 20 |
| _HEXYLSALICYLAT | 15 | 15 | 15 |
| _HEXYLZIMTALDEHYD ALPHA | 90 | 90 | 90 |
| _ISOAMYLACETAT | 2 | 2 | 2 |
| _ISOAMYLBUTYRAT | 8 | 8 | 8 |
| _JASMAPRUNAT | 15 | 15 | 15 |
| _MACROLIDE ® SUPRA | 8 | 8 | 8 |
| _MANZANATE | 4 | 4 | 4 |
| _METHYLANTHRANILAT | 1 | 1 | 1 |
| _METHYLCINNAMAT | 1.5 | 1.5 | 1.5 |
| _MUGETANOL | 1 | 1 | 1 |
| _PHENIRAT ® | 22 | 22 | 22 |
| _PRENYLACETAT | 12 | 12 | 12 |
| _ROMANDOLIDE | 15 | 15 | 15 |
| _VERTOCITRAL | 12 | 12 | 12 |
| cyclopentyl cyclohexanecarboxylate | | 40 | 60 |

According to an expert panel, the fragrance mixture without cyclopentyl cyclohexanecarboxylate smells fruity and like apple. The addition of cyclopentyl cyclohexanecarboxylate to the fragrance mixture in smaller amounts (40 parts) makes the fragrance mixture more fruity and gives an extra orange effect. The addition of cyclopentyl cyclohexanecarboxylate to the fragrance mixture in greater amounts (60 parts) makes the fragrance mixture even stronger fruity with an additional sweetness note.

1. Use of at least one compound of Formula I 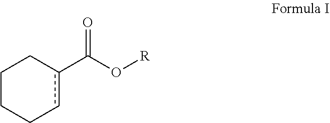
as a fragrance, wherein
R represents ethyl, propyl, butyl or cyclopentyl; and
the dotted line represents a carbon-carbon single bond or a carbon-carbon double bond.

2. Use according to claim 1, wherein R represents ethyl, butyl or cyclopentyl.

3. Use according to claim 1 and/or 2, wherein the compound of Formula I is ethyl cyclohexene-1-carboxylate.

4. Use according to claim 1 and/or 2, wherein the compound of Formula I is butyl cyclohexenecarboxylate.

5. Use according to claim 1 and/or 2, wherein the compound of Formula I is cyclopentyl cyclohexanecarboxylate.

6. Use according to claims 1 to 5 in fragrance compositions to obtain at least one of the following fragrance notes: cinnamon, marzipan, fruity, valerate, sweet, apple, pear, pineapple, strawberry, green and/or castoreum.

7. Use according to claims 1 to 6 for imparting, modifying and/or enhancing a fragrance composition to make it more fruity, juicier, softer, clearer, fresher, sweeter and/or more floral.

8. Fragrance composition comprising or consisting of at least one compound of Formula I 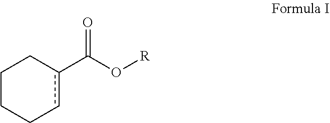
wherein
R represents ethyl, propyl, butyl or cyclopentyl; and
the dotted line represents a carbon-carbon single bond or a carbon-carbon double bond; and
at least one further fragrance.

9. Fragrance composition according to claim 8, wherein the weight ratio of the at least one compound of Formula I to the total amount of other fragrances is in the range of 1:100 to 1:10.

10. Fragrance composition according to claim 9, wherein the weight ratio of the at least one compound of Formula I to the total amount of other fragrances is in the range of 1:85 to 1:15.

11. Fragrance composition according to claims 8 to 10, wherein the amount of the at least one compound of Formula I is in the range of 0.01 to 10% by weight, based on the total weight of the fragrance composition.

12. Fragrance composition according to at least one of claims 8 to 11, the at least one compound of Formula I is contained in the fragrance composition in a sensory effective amount sufficient to modify an existing fragrance to make it more fruity, juicier, softer, clearer, fresher, sweeter and/or more floral.

13. Use of a fragrance composition according to any one of claims 8 to 12 to modify an existing fragrance to make it more fruity, juicier, softer, clearer, fresher, sweeter and/or more floral.

14. Method for modifying an existing fragrance to make it more fruity, juicier, more pineapple, softer and/or more floral,
comprising or consisting of the following steps:
(a) providing at least one compound of Formula I 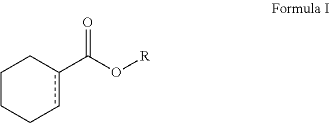
wherein
R represents ethyl, propyl, butyl or cyclopentyl; and
the dotted line represents a carbon-carbon single bond or a carbon-carbon double bond; and
(b) providing at least one further fragrance,
(c) adding the at least one compound of Formula (I) to the at least one further fragrance in a sensory effective amount sufficient to modify an existing fragrance to make it more fruity, juicier, softer, clearer, fresher, sweeter and/or more floral.

15. Perfumed product comprising at least one compound of Formula I 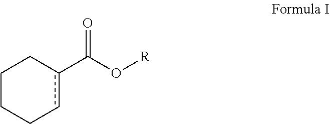
wherein
R represents ethyl, propyl, butyl or cyclopentyl; and
the dotted line represents a carbon-carbon single bond or a carbon-carbon double bond; and
two or more other fragrances and optionally other ingredients, characterized in that the product comprises the at least one compound of Formula (I) in a quantity which modifies the olfactory properties of the two or more other fragrances to make it more fruity, juicier, softer, clearer, fresher, sweeter and/or more floral.
